(19) United States
(12) Patent Application Publication
(10) Pub. No.: US 2005/0234519 A1
(43) Pub. Date: Oct. 20, 2005

(54) Cardiac stimulation device and method for automatic lower pacing rate optimization

(76) Inventors: Paul D. Ziegler, Minneapolis, MN (US); Douglas A. Hettrick, Blaine, MN (US); Rahul Mehra, Stillwater, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/824,898

(22) Filed: Apr. 15, 2004

(51) Int. Cl. A61N 1/37

(52) U.S. Cl. 607/27

(57) ABSTRACT

A method and device for determining an optimal lower rate and adjusting the programmed lower pacing rate to the optimal rate that includes monitoring a parameter in response to therapy delivered at a first rate during a first time period to generate first parameter data, and determining whether the therapy was delivered for a predetermined portion of the first time period. The parameter is monitored in response to the therapy delivered at a next rate during a next time period to generate next parameter data, and a determination is made as to whether the therapy was delivered for a predetermined portion of the next time period. A metric corresponding to the first parameter data is determined to generate a first parameter metric, and corresponding to the next parameter data to generate a next parameter metric used for determining an optimal therapy delivery rate.

### FIELD OF THE INVENTION

The present invention relates generally to implantable cardiac electrical stimulation devices and, more particularly, to a device and method for automatically determining an optimal lower pacing rate.

### BACKGROUND OF THE INVENTION

Implantable cardiac stimulation devices automatically modify the cardiac pacing rate in various applications. Increases in pacing rate above a programmed lower rate (LR), also referred to as “base rate,” are performed in response to activity or other metabolic sensors in rate response pacing to meet the metabolic demands of the patient. Overdrive pacing is used to prevent arrhythmias by pacing at a variable rate greater than the sensed intrinsic cardiac rate. Hysteresis has been provided to allow pacing to be suspended with the return of intrinsic depolarizations. However, in general, any pacing modality typically restores a nominal lower pacing rate after modifying the pacing rate for rate response, arrhythmia prevention, or other purposes.

Some evidence exists, however, in support of the concept that an optimal lower rate may exist for a particular patient in achieving a desired effect. The inventors of the present invention have observed that in some patients continuous atrial overdrive pacing at a particular rate effectively prevents atrial arrhythmias whereas atrial overdrive pacing that steps back down to a nominal lower rate results in a return of premature atrial contractions (PACs) and/or atrial tachyarrhythmias. Research reports suggest an individual's heart rate is set to match the aortic input impedance to allow the greatest cardiac efficiency. In patients that are pacemaker dependent or suffering from heart failure, the heart may perform better hemodynamically at a particular lower rate.

While considerable effort has been made optimizing other pacing parameters including pacing site, the delay between pacing sites, and pacing mode, optimization of the lower pacing rate for achieving a desired therapeutic effect has not been fully addressed previously. A system and method is needed, therefore, for determining an optimal lower pacing rate and automatically maintaining the base pacing rate at the optimal rate to achieve a desired therapeutic effect. In particular, a method for maintaining a permanent lower pacing rate at a rate that results in a minimal number of arrhythmia events is desirable.

### BRIEF DESCRIPTION OF THE DRAWINGS

Various aspects and features of the present invention will be readily appreciated as the same becomes better understood by reference to the following detailed description when considered in connection with the accompanying drawings, in which like reference numerals designate like parts throughout the figures thereof and wherein:

FIG. 1 is an illustration of an implantable cardiac stimulation device coupled to a patient's heart by way of three leads;

FIG. 2 is a functional block diagram of the implantable cardiac stimulation device of FIG. 1, in which the present invention may usefully be practiced;

FIG. 3 is an exemplary flow chart of a method for controlling therapy delivery in accordance with an embodiment of the present invention;

FIG. 4 is an illustrative example of a timing diagram showing the application of a number of test lower rates during the iterative procedure included in the method of FIG. 3 according to a testing algorithm according to the present invention;

FIG. 5 is a flow chart summarizing steps included in the iterative procedure shown in FIG. 3 when applied for optimizing the lower pacing rate for arrhythmia prevention;

FIG. 6 is a graph of sample results reporting the percentage of atrial cycles that were classified as premature atrial contractions (PACs) for a number of test pacing cycle lengths;

FIG. 7 is a flow chart summarizing a method for automatically maintaining the lower rate at an optimal rate according to an embodiment of the present invention.

### DETAILED DESCRIPTION OF THE INVENTION

The present invention addresses the above-described needs by providing a cardiac stimulation system and associated method for automatically determining an optimal lower rate and adjusting the permanent lower pacing rate to the optimal rate. The method for determining an optimal lower rate is an iterative procedure involving: delivering pacing pulses at a number of test pacing rates wherein each test pacing rate is applied for a predetermined test period duration; repeating application of all test pacing rates for the test period duration a predetermined number of times; measuring a physiological parameter during the application of each test pacing rate; performing statistical analysis of the physiological parameter data to determine a metric of the effect of pacing rate on the physiological parameter; and determining the optimal pacing rate as the test pacing rate that achieves the greatest desired effect on the measured physiological parameter.

In one embodiment, the optimal lower rate for arrhythmia prevention is determined. During the application of each pacing rate, the number of arrhythmia events, which may include premature contractions, tachycardia, fibrillation, and/or pacing mode switches, are counted. A metric of the effect of LR on arrhythmia incidence is computed as a weighted count of arrhythmia events. The optimal lower rate is determined as the rate during which the minimum weighted count occurs.

In other embodiments, the optimal lower pacing rate for achieving a maximal or desired hemodynamic or metabolic effect may be determined. A metric for identifying the lower rate during which a desired hemodynamic or metabolic effect occurs may be computed from a physiological sensor signal, which may be an electrical, mechanical or biochemical signal.

The present invention is realized in a cardiac stimulation device coupled to electrodes and any other sensor(s) needed for measuring a physiological parameter of interest. The cardiac stimulation device includes a control system for controlling device functions and executing the lower rate optimization method; a sensing interface for sensing physiological signals;

signal processing circuitry for deriving measurements of a physiological parameter from the physiological signal; and pacing timing and control circuitry and pacing output circuitry for delivering pacing pulses to the heart. The control system may execute the method for determining an optimal lower rate on a periodic or triggered basis and reset the permanent lower rate to the determined optimal lower rate whenever a new optimal lower rate is found.

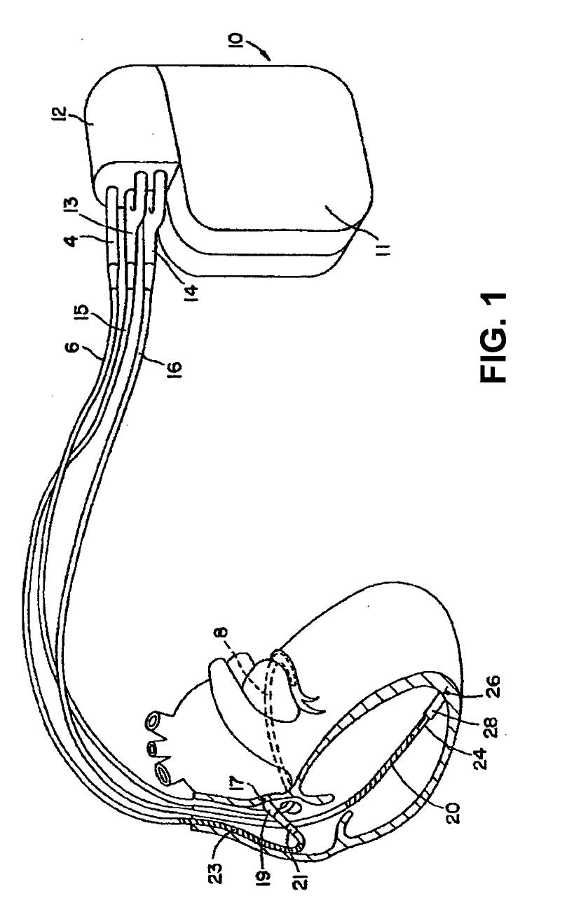

FIG. 1 is an illustration of an implantable cardiac stimulation device 10 coupled to a patient's heart by way of three leads 6, 15, and 16. A connector block 12 receives the proximal end of a right ventricular lead 16, a right atrial lead 15 and a coronary sinus lead 6, used for positioning electrodes for sensing and stimulation in three or four heart chambers. In FIG. 1, the right ventricular lead 16 is positioned such that its distal end is in the right ventricle (RV) for sensing right ventricular cardiac signals and delivering pacing or shocking pulses in the right ventricle. For these purposes, right ventricular lead 16 is equipped with a ring electrode 24, a tip electrode 26, optionally mounted retractably within an electrode head 28, and RV coil electrode 20, each of which are connected to an insulated conductor contained within the body of lead 16. The proximal end of the insulated conductors are coupled to corresponding connectors carried by connector 14 at the proximal end of lead 16 for providing electrical connection to device 10.

The right atrial lead 15 is positioned such that its distal end is in the vicinity of the right atrium and the superior vena cava (SVC). Lead 15 is equipped with a ring electrode 21 and a tip electrode 17, optionally mounted retractably within electrode head 19, for sensing and pacing in the right atrium. Lead 15 is further equipped with an SVC coil electrode 23 for delivering high-energy shock therapy. The ring electrode 21, the tip electrode 17 and the SVC coil electrode 23 are each connected to an insulated conductor within the body of the right atrial lead 15. Each insulated conductor is coupled at its proximal end to connector 13.

The coronary sinus lead 6 is advanced within the vasculature of the left side of the heart via the coronary sinus and great cardiac vein. The coronary sinus lead 6 is shown in the embodiment of FIG. 1 as having a defibrillation coil electrode 8 that may be used in combination with either the RV coil electrode 20 or the SVC coil electrode 23 for delivering electrical shocks for cardioversion and defibrillation therapies. In other embodiments, coronary sinus lead 6 may also be equipped with a distal tip electrode and ring electrode for pacing and sensing functions in the left chambers of the heart. The coil electrode 8 is coupled to an insulated conductor within the body of lead 6, which provides connection to the proximal connector 4.

The electrodes 17 and 21 or 24 and 26 may be used for pacing and sensing in bipolar pairs, commonly referred to as a “tip-to-ring” configuration, or individually in a unipolar configuration with the device housing 11 serving as the indifferent electrode, commonly referred to as the “can” or “case” electrode. The device housing 11 may also serve as a subcutaneous defibrillation electrode in combination with one or more of the defibrillation coil electrodes 8, 20 or 23 for defibrillation of the atria or ventricles.

The depicted positions of the leads and electrodes shown in FIG. 1 in or about the right and left heart chambers are approximate and merely exemplary. The present invention may be practiced using alternative lead systems having pacing/sensing electrodes adapted for placement at pacing or sensing sites in operative relation to the one or more heart chambers. Such systems may include transvenous leads as shown in FIG. 1 or may alternatively include leads having epicardial or subcutaneous electrodes. The implementation may also include a device that does not employ pacing leads as described here to detect and treat arrhythmias. For example, a device implanted subcutaneously or sub-muscularly in a position over the heart such as an axillary location could use non-intracardiac lead based methods of electrical sensing to sense cardiac activity and deliver electrical stimulation pulses. While a particular multi-chamber cardiac stimulation device and lead system is illustrated in FIG. 1, methodologies included in the present invention may be adapted for use with other single chamber, dual chamber, or multichamber cardiac stimulation systems.

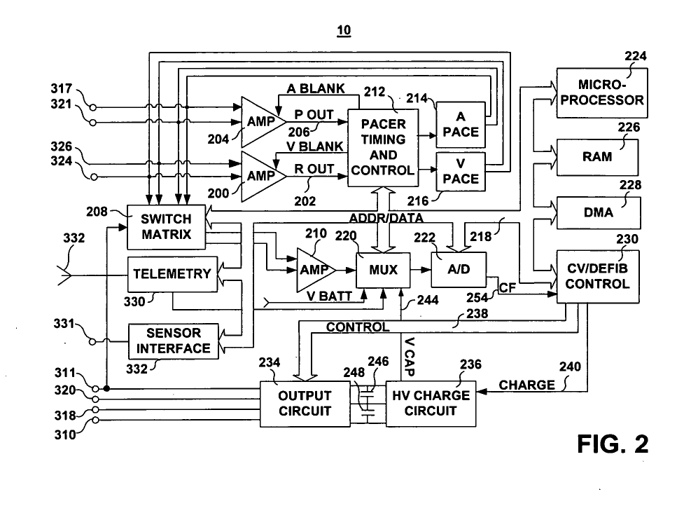

FIG. 2 is a functional block diagram of the implantable cardiac stimulation device of FIG. 1, in which the present invention may usefully be practiced. This diagram should be taken as exemplary of the type of device with which the invention may be embodied and not as limiting, as it is believed that the invention may usefully be practiced in a wide variety of device implementations. For example, devices employing the present invention will generally deliver cardiac pacing therapies, which may include bradycardia pacing, overdrive pacing, and anti-tachycardia pacing, but may or may not include cardioversion/defibrillation therapies. The disclosed embodiment shown in FIG. 2 is a microprocessor-controlled device, but the methods of the present invention may also be practiced with devices employing dedicated integrated circuitry for controlling some device functions.

With regard to the electrode system illustrated in FIG. 1, the device 10 is provided with a number of connection terminals for achieving electrical connection to the cardiac leads 6, 15, and 16 and their respective electrodes. The connection terminal 311 provides electrical connection to the housing 11 for use as the indifferent electrode during unipolar stimulation or sensing. The connection terminals 320, 310, and 318 provide electrical connection to coil electrodes 20, 8 and 23 respectively. Each of these connection terminals 311, 320, 310, and 318 are coupled to the high voltage output circuit 234 to facilitate the delivery of high energy shocking pulses to the heart using one or more of the coil electrodes 8, 20, and 23 and optionally the housing 11.

The connection terminals 317 and 321 provide electrical connection to the tip electrode 17 and the ring electrode 21 positioned in the right atrium. The connection terminals 317 and 321 are further coupled to an atrial sense amplifier 204 for sensing atrial signals such as P-waves. The connection terminals 326 and 324 provide electrical connection to the tip electrode 26 and the ring electrode 24 positioned in the right ventricle. The connection terminals 326 and 324 are further coupled to a ventricular sense amplifier 200 for sensing ventricular signals.

The atrial sense amplifier 204 and the ventricular sense amplifier 200 preferably take the form of automatic gain controlled amplifiers with adjustable sensing thresholds. The general operation of the ventricular sense amplifier 200 and the atrial sense amplifier 204 may correspond to that disclosed in U.S. Pat. No. 5,117,824, by Keimel, et al., incorporated herein by reference in its entirety. Whenever a signal received by atrial sense amplifier 204 exceeds an atrial sensing threshold, a signal is generated on the P-out signal line 206. Whenever a signal received by the ventricular sense amplifier 200 exceeds a ventricular sensing threshold, a signal is generated on the R-out signal line 202.

Switch matrix 208 is used to select which of the available electrodes are coupled to a wide band amplifier 210 for use in digital signal analysis. Selection of the electrodes is controlled by the microprocessor 224 via data/address bus 218. The selected electrode configuration may be varied as desired for the various sensing, pacing, cardioversion and defibrillation functions of the device 10. Signals from the electrodes selected for coupling to bandpass amplifier 210 are provided to multiplexer 220, and thereafter converted to multi-bit digital signals by A/D converter 222, for storage in random access memory 226 under control of direct memory access circuit 228. Microprocessor 224 may employ digital signal analysis techniques to characterize the digitized signals stored in random access memory 226 to recognize and classify the patient's heart rhythm employing any of the numerous signal processing methods known in the art.

In some embodiments, device 10 may include physiological sensor interface circuitry 332 for receiving, conditioning, and processing a signal from a physiological sensor 331. Sensor 331 is provided for sensing a physiological signal related to cardiac hemodynamic function or a metabolic state. Sensor 331 may be deployed on an intracardiac, transvenous lead, or be located in the thoracic cavity, submuscularly or subcutaneously, or on or within device 10 itself. Sensor 331 may sense electrical, mechanical or biochemical signals that may be correlated to a physiological parameter of interest by signal processing algorithms performed by dedicated circuitry included in interface 332 or by signal processing algorithms executed by microprocessor 224 after receiving signal data on bus 218. Examples of lead-based physiological sensors that may be used in conjunction with an implantable cardiac stimulation device are disclosed in U.S. Pat. No. 5,564,434 issued to Halperin et al., U.S. Pat. No. 5,535,752 issued to Halperin et al., and U.S. Pat. No. 4,750,495 issued to Moore and Brumwell, all of which are incorporated herein by reference in their entirety. In accordance with one embodiment of the present invention, a physiological sensor signal may be monitored for determining a physiological parameter used for identifying an optimal lower pacing rate according to the effect of the pacing rate on the physiological parameter.

The telemetry circuit 330 receives downlink telemetry from and sends uplink telemetry to an external programmer, as is conventional in implantable anti-arrhythmia devices, by means of an antenna 332. Received telemetry is provided to microprocessor 224 via multiplexer 220. Data to be uplinked to the programmer and control signals for the telemetry circuit 330 are provided by microprocessor 224 via address/data bus 218. Data to be uplinked may include a record of detected arrhythmia episodes, physiological data or other patient-related or device-related data as is customary in modern implantable cardiac stimulation/monitoring devices. Numerous types of telemetry systems known for use in implantable medical devices may be used.

The remainder of circuitry illustrated in FIG. 2 is dedicated to the provision of cardiac pacing, cardioversion and defibrillation therapies and, for the purposes of the present invention, may correspond to circuitry known in the prior art. In the exemplary embodiment shown in FIG. 2, the pacer timing and control circuitry 212 includes programmable digital counters which control the basic time intervals associated with various single, dual or multi-chamber pacing modes or anti-tachycardia pacing therapies delivered in the atria or ventricles. Pacer circuitry 212 also determines the amplitude of the cardiac pacing pulses under the control of microprocessor 224.

During pacing, escape interval counters within pacer timing and control circuitry 212 are reset upon sensing of R-waves or P-waves as indicated by signals on lines 202 and 206, respectively. In accordance with the selected mode of pacing, pacing pulses are generated by atrial pacer output circuit 214 and/or ventricular pacer output circuit 216. The pacer output circuits 214 and 216 are coupled to the desired electrodes for pacing via switch matrix 208. The escape interval counters are reset upon generation of pacing pulses, and thereby control the basic timing of cardiac pacing functions, including anti-tachycardia pacing.

The durations of the escape intervals are determined by microprocessor 224 via data/address bus 218. In accordance with the present invention, microprocessor 224 will determine an optimal lower rate for setting a base rate escape interval as will be described in greater detail below. The value of the count present in the escape interval counters when reset by sensed R-waves or P-waves can be used to measure R-R intervals, P-P intervals, P-R intervals, and R-P intervals, which measures are stored in memory 226 and to diagnose the occurrence of a variety of arrhythmias.

Microprocessor 224 operates as an interrupt driven device and is responsive to interrupts from pacer timing and control circuitry 212 corresponding to the occurrences of sensed P-waves and R-waves and corresponding to the generation of cardiac pacing pulses. Any necessary mathematical calculations to be performed by microprocessor 224 and any updating of the values or intervals controlled by pacer timing/control circuitry 212 take place following such interrupts. These calculations include those described in more detail below associated with the lower rate optimization methods included in the present invention.

A portion of the random access memory 226 may be configured as a number of recirculating buffers capable of holding a series of measured intervals, which may be analyzed in response to a pace or sense interrupt by microprocessor 224 for diagnosing an arrhythmia. In response to the detection of atrial or ventricular tachycardia, an anti-tachycardia pacing therapy may be delivered if desired by loading a regimen from microcontroller 224 into the pacer timing and control circuitry 212 according to the type of tachycardia detected. Alternatively, circuitry for controlling the timing and generation of anti-tachycardia pacing pulses as generally described in U.S. Pat. No. 4,577,633 issued to Berkovits et al., U.S. Pat. No. 4,880,005 issued to Pless et al., U.S. Pat. No. 4,726,380 issued to Vollmann et al., and U.S. Pat. No. 4,587,970 issued to Holley et al., all of which patents are incorporated herein by reference in their entireties, may be used.

In the event that higher voltage cardioversion or defibrillation shock pulses are required, microprocessor 224 activates the cardioversion and defibrillation control circuitry 230 to initiate charging of the high voltage capacitors 246 and 248 via charging circuit 236 under the control of high voltage charging control line 240. The voltage on the high voltage capacitors 246 and 248 is monitored via a voltage capacitor (VCAP) line 244, which is passed through the multiplexer 220. When the voltage reaches a predetermined value set by microprocessor 224, a logic signal is generated on the capacitor full (CF) line 254, terminating charging. Thereafter, timing of the delivery of the defibrillation or cardioversion pulse is controlled by pacer timing and control circuitry 212.

One embodiment of an appropriate system for delivery and synchronization of ventricular cardioversion and defibrillation pulses and for controlling the timing function related to them is generally disclosed in commonly assigned U.S. Pat. No. 5,188,105 to Keimel, incorporated herein by reference in its entirety. If atrial defibrillation capabilities are included in the device, appropriate systems for delivery and synchronization of atrial cardioversion and defibrillation pulses and for controlling the timing function related to them may be found in U.S. Pat. No. 4,316,472 issued to Mirowski et al., U.S. Pat. No. 5,411,524 issued to Mehra, or U.S. Pat. No. 6,091,988 issued to Warman, all of which patents are incorporated herein by reference in their entireties. Any known ventricular cardioversion or defibrillation pulse control circuitry may be usable in conjunction with the present invention. For example, circuitry controlling the timing and generation of cardioversion and defibrillation pulses as disclosed in U.S. Pat. No. 4,384,585, issued to Zipes, U.S. Pat. No. 4,949,719, issued to Pless et al., and in U.S. Pat. No. 4,375,817, issued to Engle et al., all incorporated herein by reference in their entireties may be used in a device employing the present invention.

In the illustrated device, delivery of cardioversion or defibrillation pulses is accomplished by output circuit 234, under control of control circuitry 230 via control bus 238. Output circuit 234 determines the shock pulse waveform, e.g. whether a monophasic, biphasic or multiphasic pulse is delivered, whether the housing 311 serves as cathode or anode, which electrodes are involved in delivery of the pulse, and the pulse shape and tilt. Examples of high-voltage cardioversion or defibrillation output circuitry are generally disclosed in U.S. Pat. No. 4,727,877 issued to Kallok, and U.S. Pat No. 5,163,427 issued to Keimel, both incorporated herein by reference in their entirety.

Examples of output circuitry for delivery of biphasic pulse regimens may be found in U.S. Pat. No. 5,261,400 issued to Bardy, and U.S. Pat. No. 4,953,551 issued to Mehra et al., incorporated herein by reference in its entirety. An example of circuitry which may be used to control delivery of monophasic pulses is set forth in the above cited U.S. Pat. No. 5,163,427, to Keimel. However, output control circuitry for generating a multiphasic defibrillation pulse as generally disclosed in U.S. Pat. No. 4,800,883, issued to Winstrom, incorporated herein by reference in its entirety, may also be used in conjunction with a device embodying the present invention.

In modern implantable cardioverter defibrillators, the particular therapies are programmed into the device ahead of time by the physician, and a menu of therapies is typically provided. For example, on initial detection of tachycardia, an anti-tachycardia pacing therapy may be selected. On redetection of tachycardia, a more aggressive anti-tachycardia pacing therapy may be scheduled. If repeated attempts at anti-tachycardia pacing therapies fail, a higher-level cardioversion pulse therapy may be selected thereafter. As in the case of currently available implantable cardioverter defibrillators (ICDs), and as discussed in the above-cited references, the amplitude of the defibrillation shock may be incremented in response to failure of an initial shock or shocks to terminate fibrillation. Prior art patents illustrating such pre-set therapy menus of anti-tachycardia therapies include the above-cited U.S. Pat. No. 4,726,380 issued to Vollmann et al., above cited U.S. Pat. No. 4,587,970 issued to Holley et al., and U.S. Pat. No. 4,830,006 issued to Haluska, incorporated herein by reference in their entirety.

The following exemplary arrhythmia detection method corresponds to that employed in implantable pacemaker/cardioverter/defibrillators and employs rate/interval based timing criteria as a basic mechanism for detecting the presence of a tachyarrhythmia. To this end, the device defines a set of rate ranges and associated software-defined counters to track the numbers of intervals falling within the defined ranges.

A first rate range may define a minimum R-R or P-P interval used for ventricular fibrillation (VF) or atrial fibrillation detection (AF), respectively, referred to as a “fibrillation detection interval” or “FDI”. An associated VF or AF count preferably indicates how many of a first predetermined number of the preceding intervals were shorter than the FDI. A second rate range may include R-R or P-P intervals shorter than a lower tachycardia detection interval “TDI”, and an associated VT count or AT count is incremented in response to an interval shorter than the TDI but longer than the FDI, is not affected by intervals shorter than the FDI, and is reset in response to intervals longer than the TDI. Optionally, the device may include a third rate range including intervals longer than the FDI interval, but shorter than a fast tachycardia interval (FTDI) which is intermediate the lower tachycardia detection interval (TDI) and the lower fibrillation detection interval (FDI).

For purposes of the present example, the interval counts may be used to signal detection of an associated arrhythmia (fibrillation, fast tachycardia or slow tachycardia) when they individually or in combination reach a predetermined value, referred to herein as “number of intervals to detect” or “NID”. Each rate zone may have its own defined count and NID, for example “AFNID” for atrial fibrillation detection and “ATNID” for atrial tachycardia detection or combined counts may be employed. These counts, along with other stored information reflective of the previous series of R-R, P-P, P-R, and R-P intervals such as information regarding the rapidity of onset, the stability of the detected intervals, the duration of continued detection of short intervals, the average interval duration and information derived from analysis of stored EGM segments are used to determine whether tachyarrhythmias are present and to distinguish between different types of tachyarrhythmias.

For purposes of illustrating the invention, an exemplary rate/interval based arrhythmia detection method is described above. Other tachyarrhythmia detection methodologies, including detection methods as described in U.S. Pat. No. 5,991,656, issued to Olson, et al., U.S. Pat. No. 5,755,736, issued to Gillberg, et al., both incorporated herein by reference in their entireties, or other known ventricular and/or atrial tachyarrhythmia detection methods may be substituted. It is believed that the method for controlling therapy delivery of the present invention may be usefully practiced in conjunction with virtually any underlying rate-based arrhythmia detection scheme. Other exemplary detection schemes are described in U.S. Pat. No. 4,726,380, issued to Vollmann, U.S. Pat. No. 4,880,005, issued to Pless et al. and U.S. Pat. No. 4,830,006, issued to Haluska et al., incorporated by reference in their entireties herein. However, other criteria may also be measured and employed in conjunction with the present invention.

Criteria for detecting premature contractions may also be event interval based. For example, premature ventricular contractions (PVCs) may be based on the detection of two ventricular events in a row without an intervening atrial event. Detection of runs of premature atrial contractions (PACs) may be based on sensing alternating short and long P-P intervals while isolated PACs may be detected when two successive atrial events are sensed without an intervening ventricular event or when a measured P-P interval is less than a running median or mean P-P interval.

For purposes of the present invention, the particular details of implementation of the rate/interval based arrhythmia detection methodologies are not of primary importance. However, in applications for controlling therapy delivery for arrhythmia prevention, it is required that the rate based detection methodologies employed by the device allow identification and detection of rhythms representing an arrhythmia, which may include premature beats, for example. The number and type of arrhythmia detections made during application of the method for controlling therapy delivery will be used in determining an optimal lower rate for arrhythmia prevention, as will be described in greater detail below.

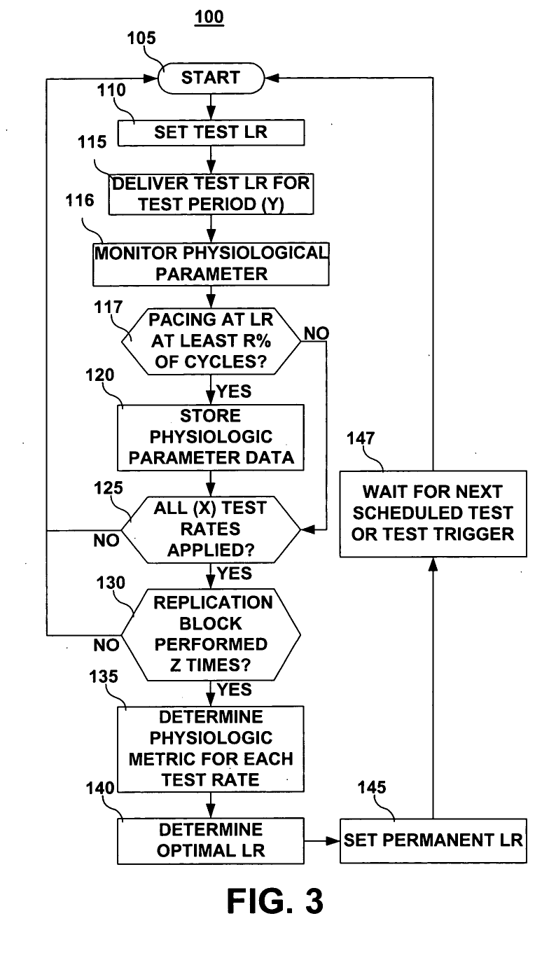

FIG. 3 is an exemplary flow chart of a method for controlling therapy delivery in accordance with an embodiment of the present invention. Method 100 is an iterative procedure for applying a number of test lower rates for a test period during which the effect of the lower rate is measured by monitoring a physiological parameter or physiological events. Method 100 is expected to be particularly beneficial in determining an optimal lower rate for the prevention of atrial arrhythmias, for example, however method 100 may be useful in determining an optimal lower rate for the prevention of ventricular arrhythmias and may even be used for determining an optimal lower rate for achieving hemodynamic-related benefits.

Method 100 may be implemented in software/firmware resident in microprocessor 224 of device 10. Method 100 may be activated by a clinician or programmed to be activated at a particular time and/or date. Method 100 may also be enabled to run on a periodic basis, e.g., weekly or monthly. The frequency of repeating method 100 for re-determining an optimal lower rate may depend on the physiological effect being optimized. In one embodiment, method 100 may be executed with greater frequency early on and with decreasing frequency thereafter, e.g., once a week for a period of one month, and thereafter once a month for one year, etc.

After being activated at step 105, the lower rate is set at a test lower rate at step 110. A number of test lower rates are set beforehand by a clinician or may be selected according to a default set of test lower rates. For example, lower rates ranging from 60 to 85 bpm in 5 bpm increments may be tested. The test lower rate set at step 110 is applied for a predetermined test period duration at step 115. The test period duration may be selected by a clinician and may range from one to a few minutes, one to a few hours or even one to a few days. In arrhythmia prevention applications, the test period duration will typically be on the order of hours, for example one hour.

During the test period, a physiological parameter is monitored at step 116. After the test period duration has expired, method 100 determines at decision step 117 if the amount of time or the number of cardiac cycles for which device 10 was pacing at the test lower rate during the test period meets a lower rate pacing requirement. Preferably, pacing at the test lower rate is occurring a majority of the time during a test period in order to accurately assess the effect of the test rate on the physiological parameter of interest. For example, pacing at the test LR at least a predetermined percentage (R %) of time or a predetermined percentage of all cardiac cycles, e.g., at least 70% of the time or 70% of all cardiac cycles, may be required for the test period to be considered valid for evaluating the effect of the test lower rate on the physiological parameter of interest.

If the test period is determined to be valid based on the lower rate pacing requirement being met at decision step 117, monitored physiological parameter data is stored at step 120. Physiological parameter data that may be stored may be a measure of the number and type of arrhythmia events, which may include premature contractions, tachycardia episodes and fibrillation episodes detected during application of the test lower rate. Arrhythmia-related events, such as pacing mode switches may also be counted. In other embodiments, a physiological parameter that may be monitored and for which data is stored at step 120 may be a hemodynamic or metabolic parameter measured from a physiological sensor.

If a test period is determined to be invalid based on insufficient pacing at the test lower rate as determined at decision step 117, the physiological parameter data for that test period is not recorded at step 120. Rather, method 100 proceeds directly to step 125 to determine if all test lower rates have been applied and found to be valid at decision step 125. Any remaining test lower rates are applied by returning to step 100 to set the lower rate to the next test rate. Invalid test periods may be repeated either immediately or after all other test lower rates have been applied, thereby extending the duration of the total testing period.

Once all test lower rates have been applied for the test period (and each test period determined to be valid for storing physiological parameter data), method 100 determines if a replication block has been performed a desired number of times, Z. A replication block consists of all the selected test lower rates, each applied for the test period. The replication block may be repeated one or more times such that each test lower rate is applied for a test period Z times. Within each replication block, the order in which the test rates are applied may be altered or randomized such that the test rates are applied in a different order within each replication block. Preferably, each test rate is repeated at different times of day so that circadian effects, patient activity, or other time-related factors do not bias the results. The total duration of time required to perform the iterative procedure may be calculated as the number of test lower rates (X) multiplied by the test period duration (Y) and the number of replication blocks (Z).

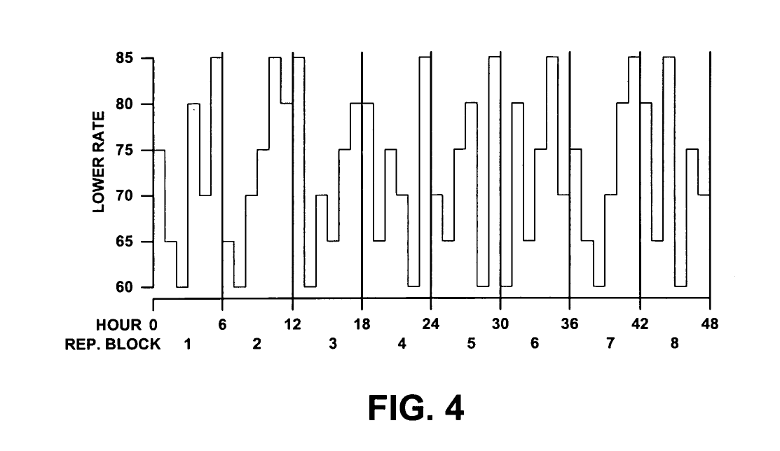

FIG. 4 is a timing diagram illustrating the application of a number of test lower rates during the iterative procedure of method 100 in a testing algorithm according to the present invention. Six test lower rates ranging from 60 to 85 bpm in 5 bpm increments are tested. Each test lower rate is applied for a test period of one hour within each replication block. Eight replication blocks are performed such that the total time required to perform the iterative procedure is 48 hours (test duration=X*Y*Z=6*1*8=48 hours). Within each replication block, the order that the test lower rates are applied is randomized. In the example shown, a rate of 75 bpm will be applied during hour 1, hour 10, hour 17, hour 21, hour 27, hour 34, hour 37, and hour 47. By randomizing the order of the test rates, the test rates will be applied at different times of day to eliminate diurnal effects.

Changes between test rates are shown in FIG. 4 to be abrupt, single step changes. However, in implementation it is recognized that gradual, multi-step or smoothed transitions between test rates are preferred. Sudden large step changes in pacing rate may have deleterious hemodynamic or arrhythmogenic consequences. Transition time between each test rate may therefore extend the total test duration when smoothed, multi-step rate changes are made. Furthermore, it may be desirable to delay monitoring of a physiological parameter until a steady-state response to a new test lower rate has been reached. Monitoring may be continuous through transitions between test rates however physiological parameter data acquired during a rate transition phase may be excluded from computations made for determining an optimal lower rate.

Once all replication blocks have been executed, as determined at decision step 130 of FIG. 3, a metric of the physiological parameter(s) measured at step 120 is computed at step 135 for each test rate. As will be described in greater detail below, in arrhythmia prevention applications, a metric computed at step 135 may relate to the number and type of arrhythmic events detected during each test period. In other applications, a hemodynamic or metabolic parameter may be averaged or otherwise statistically analyzed to determine a metric of the hemodynamic or metabolic parameter for each test period. Data acquired during each test period for a given test rate may be processed separately to determine a metric for each test period and then the metrics for each test period statistically processed to determine an overall metric for each test rate. Alternatively, all data acquired during all test periods for a given test rate may be combined for computing a metric for the test rate.

After computing a metric for each test rate, an optimal lower rate is determined at step 140. The optimal lower rate is identified as the test rate having the greatest desired effect on the physiologic metric computed at step 135. At step 145, the permanent lower pacing rate may be automatically programmed to the optimal lower rate. Alternatively, optimal lower rate data may be stored in device memory for review by a clinician, at which time the permanent lower rate may be programmed manually to an optimal lower rate selected based on clinician review of the test results.

At step 147, method 100 waits for the next scheduled test time or for a test trigger to occur. A lower rate optimization test may be initiated manually by a clinician or automatically on a scheduled basis as described previously. Additionally or alternatively, method 100 may be automatically triggered based on previously-defined trigger criteria. For example, the physiological parameter monitored during execution of method 100 may be monitored continuously or periodically for computation of a metric of the physiological parameter. If the metric crosses a threshold or changes by more than a predetermined amount, the optimal lower rate may have changed due to a change in disease state, patient medication, or other physiological change. For example, in arrhythmia-prevention applications, if the number of arrhythmia events detected during a specified interval of time increases by more than an predefined amount or crosses a predefined threshold, method 100 may be triggered to allow a re-determination of the optimal lower rate.

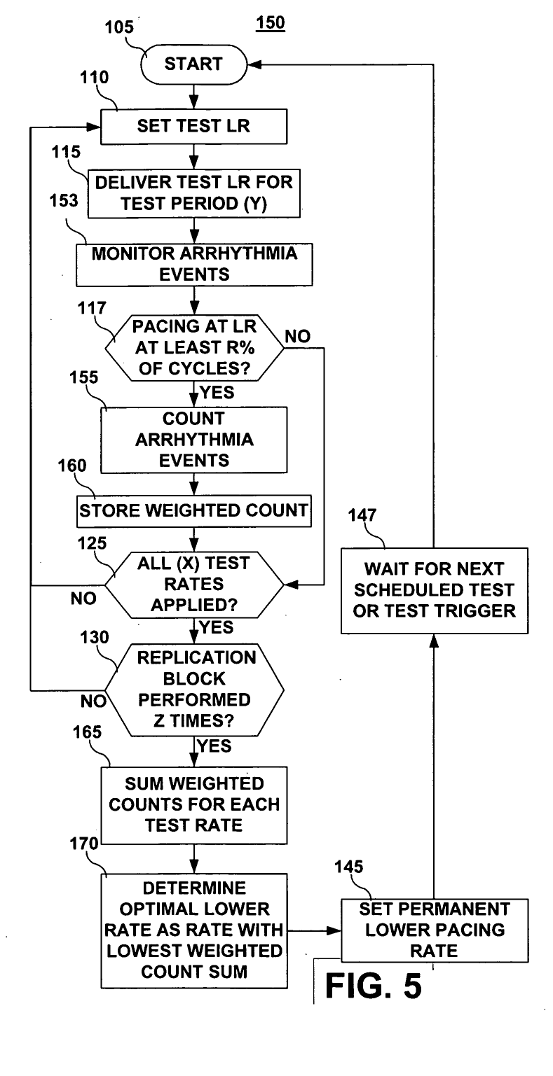

FIG. 5 is a flow chart summarizing steps included in the iterative procedure shown in FIG. 3 when applied for optimizing the lower pacing rate for arrhythmia prevention. Steps included in method 150 correspond to identically-numbered steps in method 100 described above. In method 150, however, arrhythmia events are monitored at step 153, which corresponds to step 116 of method 100 for monitoring a physiological parameter. Steps 155 and 160 correspond generally to step 120 of method 100 wherein physiologic parameter data is stored. In method 150, the physiologic parameter data to be stored is a count of the incidence of arrhythmia events, which may include premature contractions, tachycardias, fibrillation, and/or pacing mode switches. Therefore at step 155, the arrhythmia events detected during pacing at a test lower rate selected at step 110, applied for a test period duration at step 115, are counted.

At step 160, a weighted count of the detected arrhythmia events is determined and stored as the physiological parameter data. Arrhythmia events may each be assigned a weighting value based on the severity of the type of event. For example, an isolated premature contraction may be assigned a weight of 1, a run of premature contractions may be assigned a higher weight, a tachycardia may be assigned a still higher rate, and fibrillation may be assigned the highest weight. The number of each type of events is multiplied by the weighting factor and the sum of the weighted counts is stored for a given test period at step 160.

At step 165, after all replication blocks have been performed, the weighted count sums determined for each test period are summed for a given test lower rate to determine an overall weighted count sum for each test rate. Alternatively, an average or median weighted count sum may be determined for each test rate from the stored counts from each test period. At step 170, the optimal lower rate is determined as the rate having the lowest weighted count sum computed at step 165. The permanent lower rate may be automatically programmed to the optimal lower rate at step 145.

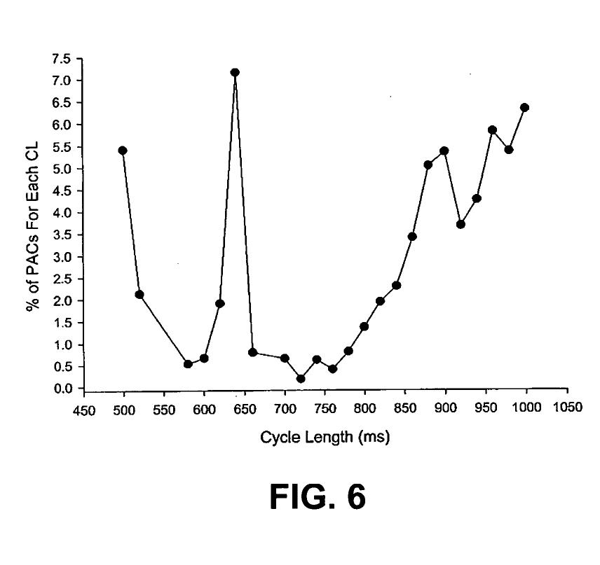

FIG. 6 is a graph of sample results reporting the percentage of atrial cycles that were classified as PACs for a number of test pacing cycle lengths. Pacing cycle lengths ranging from 500 to 1000 ms, corresponding to 120 bpm to 60 bpm, respectively, were tested in 25 ms cycle length increments. A “trough” corresponding to a reduced incidence of PACs is readily observed. Relatively long and relatively short cycle lengths resulted in considerable increases in the incidence of PACs. In this particular example, a cycle length of 725 ms, corresponding to a heart rate of approximately 83 bpm, resulted in the fewest PACS and could therefore be selected as the optimal lower rate for arrhythmia prevention.

In a method for preventing arrhythmias, therefore, the optimal lower rate is determined using method 150 described above in conjunction with FIG. 5, and the optimal lower rate is set as the permanent lower pacing rate thereafter. Constant rate overdrive pacing is delivered at the optimal lower pacing rate without altering the pacing rate (e.g., without stepping the overdrive pacing rate back down to a nominal lower rate or pacing at a variable rate slightly greater than the intrinsic rate) as is commonly done in prior art overdrive pacing techniques. Since the lower rate is the only parameter adjusted, this method for arrhythmia prevention may operate in any single or dual chamber pacing mode and may operate in combination with any other pacing algorithms that may be operating.

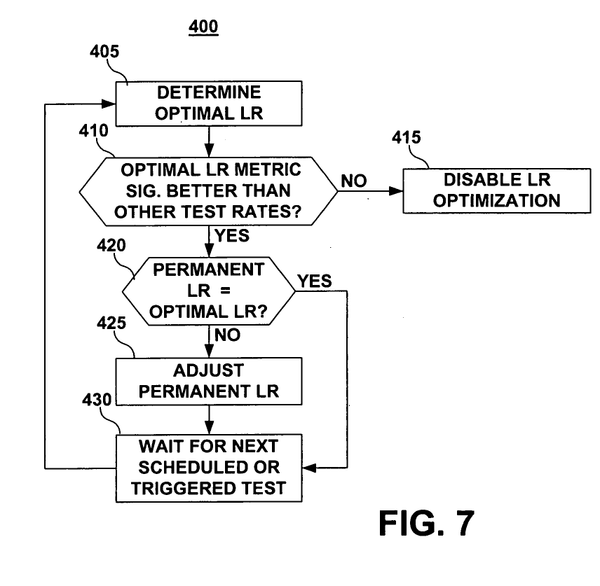

FIG. 7 is a flow chart summarizing a method for automatically maintaining the lower rate at an optimal rate. At step 405, the optimal lower rate is determined according to the iterative procedure described above. At step 410, method 400 determines if the physiological parameter metric computed for the optimal lower rate is significantly better than the metric computed for other test rates. If a significant improvement is found at decision step 410, and the current permanent lower rate is not equal to the optimal lower rate, as determined at decision step 420, the permanent rate is automatically adjusted to the optimal lower rate at step 425. After which, method 400 waits for the next scheduled or triggered optimization test at step 430. If the optimal lower rate is equal to the current permanent lower rate, as determined at decision step 420, no adjustment is necessary and method 400 proceeds directly to step 430.

According to an embodiment of the present invention, determining whether there is a significant improvement would correspond to determining whether the number of arrhythmia events detected during the test period associated with the determined optimal lower rate is at least a predetermined percentage less than the number of arrhythmia events detected during the test period associated with the other test rates. For example, if the percentage is programmed at 25%, and the optimal lower rate metric corresponding to the determined optimal lower rate corresponds to 15 arrhythmia events being detected, that optimal lower rate metric is determined to be significantly better than other test rates if those test rates correspond to arrhythmia event counts greater than or equal to 20 events (15 events is 25% less than 20 events). According to another embodiment of the present invention, the optimal lower rate metric is determined to be significantly better than other test rates if the number of events detected during the test period associated with the determined optimal lower rate is less than the number of events detected during the test period associated with the other test rates by a predetermined number, such as five, for example.

If however, the optimal lower rate identified at step 405 does not result in a significant improvement in the physiological parameter over other test rates, or in particular over the current permanent lower pacing rate, as determined at step 410, no adjustment to the permanent pacing rate will be made. Furthermore, method 400 may be designed as a self-extracting algorithm. If no significant improvement is found at step 410 for any of the test rates over each other or the current permanent pacing rate during one or more optimization tests, the lower rate optimization method may be automatically disabled at step 415. Thus, lower rate optimization and automatic permanent lower rate adjustments will continue only so long as significant improvements in the physiological parameter of interest are found.

Some of the techniques described above may be embodied as a computer-readable medium that includes instructions for a programmable processor such as microprocessor 224 or pacer timing/control circuitry 212 shown in FIG. 2. The programmable processor may include one or more individual processors, which may act independently or in concert. A “computer-readable medium” includes but is not limited to any type of computer memory such as floppy disks, conventional hard disks, CD-ROMS, Flash ROMS, nonvolatile ROMS, RAM and a magnetic or optical storage medium. The medium may include instructions for causing a processor to perform any of the features described above for actively determining a coupling interval according to the present invention.

Thus a system and method have been described for determining and maintaining an optimal lower pacing rate for achieving a desired physiological effect. The present invention has been described in detail herein according to preferred embodiments contemplated to date. It is recognized that one having skill in the art and the benefit of the teachings provided herein may conceive of numerous modifications or variations of the described embodiments. The descriptions provided herein are intended to be exemplary, therefore, and not limiting with regard to the following claims.

1. A method for controlling delivery of a therapy in an implantable medical device, comprising:
delivering a therapy at a first rate during a first time period;
monitoring a parameter in response to the therapy delivered at the first rate to generate first parameter data;
determining whether the therapy was delivered for a predetermined portion of the first time period;
delivering the therapy at a next rate during a next time period;
monitoring the parameter in response to the therapy delivered at the next rate to generate next parameter data;
determining whether the therapy was delivered for a predetermined portion of the next time period;
determining a metric corresponding to the first parameter data to generate a first parameter metric, and to the next parameter data to generate a next parameter metric; and
determining one of the first rate and the next rate as an optimal therapy delivery rate in response to the first parameter metric and the next parameter metric.

2. The method of claim 1, further comprising:
repeating delivery of the therapy at the first rate during the first time period in response to the therapy not being delivered for the predetermined portion of the first time period;
monitoring the parameter in response to the repeated delivery of the therapy at the first rate to generate updated first parameter data;
determining whether the repeated delivery of the therapy at the first rate was delivered for the predetermined portion of the first time period; and
determining a metric corresponding to the updated first parameter data to generate the first parameter metric.

3. The method of claim 2, further comprising:
repeating delivery of the therapy at the next rate during the next time period in response to the therapy not being delivered for the predetermined portion of the next time period;
monitoring the parameter in response to the repeated delivery of the therapy at the next rate to generate updated next parameter data;
determining whether the repeated delivery of the therapy at the next rate was delivered for the predetermined portion of the next time period;
determining a metric corresponding to the updated next parameter data to generate the next parameter metric.

4. The method of claim 3, wherein determining whether repeating delivery of the therapy at the first rate during the first time period and repeating delivery of the therapy at the next rate during the next time period have been repeated a predetermined number of times.

5. The method of claim 1, wherein the parameter corresponds to one of a number of arrhythmia events, a number of type of arrhythmia events, a hemodynamic event, and a metabolic event.

6. The method of claim 1, further comprising determining whether a predetermined number of arrhythmia events are detected during a predetermined time interval prior to delivering the therapy at the first rate.

7. The method of claim 1, further comprising:
delivering the therapy at the first rate during a second time period different from the first time period;
monitoring the parameter in response to the therapy delivered at the first rate during the second time period to generate second parameter data;
determining whether the therapy was delivered for a predetermined portion of the second time period;
delivering the therapy at a second next rate during a second next time period not equal to the first next time period;
monitoring the parameter in response to the therapy delivered at the second next rate to generate second next parameter data;
determining whether the therapy was delivered for a predetermined portion of the second next time period; and
determining a metric corresponding to the first parameter data and the second parameter data to generate the first parameter metric, and to the next parameter data and the second next parameter data to generate the next parameter metric.

8. The method of claim 5, wherein the first parameter data and the next parameter data correspond to a weighted count of one of the number of arrhythmia events and the number of type of arrhythmia events.

9. The method of claim 1, further comprising:
determining whether the one of the first parameter metric and the next parameter metric corresponding to the one of the first rate and the next rate determined as an optimal therapy delivery rate is less than the other of the first parameter metric and the next parameter metric by a predetermined threshold; and
setting the one of the first rate and the next rate determined as an optimal therapy delivery rate as a current therapy delivery rate only in response to the one of the first parameter metric and the next parameter metric being less than the other of the first parameter metric and the next parameter metric by the predetermined threshold.

10. A computer-readable medium having computer-executable instructions for performing a method, comprising:
delivering a therapy at a first rate during a first time period;
monitoring a parameter in response to the therapy delivered at the first rate to generate first parameter data;
determining whether the therapy was delivered for a predetermined portion of the first time period;
delivering the therapy at a next rate during a next time period;
monitoring the parameter in response to the therapy delivered at the next rate to generate next parameter data;
determining whether the therapy was delivered for a predetermined portion of the next time period;
determining a metric corresponding to the first parameter data to generate a first parameter metric, and to the next parameter data to generate a next parameter metric; and
determining one of the first rate and the next rate as an optimal therapy delivery rate in response to the first parameter metric and the next parameter metric.

11. An implantable medical device, comprising:
means for delivering a therapy at a first rate during a first time period;
means for monitoring a parameter in response to the therapy delivered at the first rate to generate first parameter data;
means for determining whether the therapy was delivered for a predetermined portion of the first time period;
means for delivering the therapy at a next rate during a next time period;
means for monitoring the parameter in response to the therapy delivered at the next rate to generate next parameter data;
means for determining whether the therapy was delivered for a predetermined portion of the next time period;
means for determining a metric corresponding to the first parameter data to generate a first parameter metric, and to the next parameter data to generate a next parameter metric; and
means for determining one of the first rate and the next rate as an optimal therapy delivery rate in response to the first parameter metric and the next parameter metric.

12. The device of claim 11, further comprising:
means for repeating delivery of the therapy at the first rate during the first time period in response to the therapy not being delivered for the predetermined portion of the first time period;
means for monitoring the parameter in response to the repeated delivery of the therapy at the first rate to generate updated first parameter data;
means for determining whether the repeated delivery of the therapy at the first rate was delivered for the predetermined portion of the first time period; and
means for determining a metric corresponding to the updated first parameter data to generate the first parameter metric.

13. The device of claim 12, further comprising:
means for repeating delivery of the therapy at the next rate during the next time period in response to the therapy not being delivered for the predetermined portion of the next time period;
means for monitoring the parameter in response to the repeated delivery of the therapy at the next rate to generate updated next parameter data;
means for determining whether the repeated delivery of the therapy at the next rate was delivered for the predetermined portion of the next time period; and
means for determining a metric corresponding to the updated next parameter data to generate the next parameter metric.

14. The device of claim 13, wherein determining whether repeating delivery of the therapy at the first rate during the first time period and repeating delivery of the therapy at the next rate during the next time period have been repeated a predetermined number of times.

15. The device of claim 11, wherein the parameter corresponds to one of a number of arrhythmia events, a number of type of arrhythmia events, a hemodynamic event, and a metabolic event.

16. The device of claim 11, further comprising means for determining whether a predetermined number of arrhythmia events are detected during a predetermined time interval prior to delivering the therapy at the first rate.

17. The device of claim 11, further comprising:
means for delivering the therapy at the first rate during a second time period different from the first time period;
means for monitoring the parameter in response to the therapy delivered at the first rate during the second time period to generate second parameter data;
means for determining whether the therapy was delivered for a predetermined portion of the second time period;
means for delivering the therapy at a second next rate during a second next time period not equal to the first next time period;
means for monitoring the parameter in response to the therapy delivered at the second next rate to generate second next parameter data;
means for determining whether the therapy was delivered for a predetermined portion of the second next time period; and
means for determining a metric corresponding to the first parameter data and the second parameter data to generate the first parameter metric, and to the next parameter data and the second next parameter data to generate the next parameter metric.

18. The device of claim 15, wherein the first parameter data and the next parameter data correspond to a weighted count of one of the number of arrhythmia events and the number of type of arrhythmia events.

19. The device of claim 11, further comprising:
means for determining whether the one of the first parameter metric and the next parameter metric corresponding to the one of the first rate and the next rate determined as an optimal therapy delivery rate is less than the other of the first parameter metric and the next parameter metric by a predetermined threshold;
means for setting the one of the first rate and the next rate determined as an optimal therapy delivery rate as a current therapy delivery rate only in response to the one of the first parameter metric and the next parameter metric being less than the other of the first parameter metric and the next parameter metric by the predetermined threshold.
